(19) United States
(12) Patent Application Publication
(10) Pub. No.: US 2022/0155323 A1
(43) Pub. Date: May 19, 2022

(54) COMPOSITIONS AND METHODS FOR DIAGNOSING ACUTE INFECTIOUS PERITONITIS

(71) Applicant: RESEARCH INSTITUTE AT NATIONWIDE CHILDREN'S HOSPITAL, Columbus, OH (US)

(72) Inventors: Michael B. BECKNELL, Westerville, OH (US); Rose M. AYOOB, South Charleston, WV (US); Neha D. POTTANAT, Indianapolis, IN (US); Matthias EBERL, Cardiff (GB)

(21) Appl. No.: 17/439,092

(22) PCT Filed: Mar. 13, 2020
(86) PCT No.: PCT/US2020/022557

§ 371 Date: Sep. 14, 2021

Related U.S. Application Data
(60) Provisional application No. 62/818,814, filed on Mar. 15, 2019.

(51) Int. Cl. G01N 33/68; G01N 33/573; C12Q 1/6883; C12Q 1/6804

(52) U.S. Cl. CPC G01N 33/6893; G01N 33/573; C12Q 1/6883; G01N 2800/06; G01N 2333/922; C12Q 2600/158; C12Q 1/6804

(57) ABSTRACT

A method of diagnosing infection in a patient undergoing a treatment of a predetermined type. This method comprises establishing a baseline level of at least one antimicrobial peptide in a specific patient or in a patient population undergoing treatment of a predetermined type, wherein the treatment involves accessing fluid from the patient's peritoneal cavity prior to the patient presenting with a suspected infection; measuring the level of the at least one antimicrobial peptide in a sample taken from the patient after the patient presents with a suspected infection; comparing the baseline level of the at least one antimicrobial peptide to the measured level of the at least one antimicrobial peptide; and diagnosing the patient with an infection if the baseline level of the at least one antimicrobial peptide exceeds the measured level of the at least one microbial peptide by a predetermined amount.

### CROSS-REFERENCE TO RELATED APPLICATIONS

This application is a national stage application filed under 35 U.S.C. § 371 of International Patent Application No. PCT/US2020/022557, filed on Mar. 13, 2020, which is incorporated by reference herein in its entirety for all purposes.

### REFERENCE TO A SEQUENCE LISTING

A sequence listing in computer readable form (CRF) is on file. The sequence listing is in an ASCII text (.txt) file entitled SEQIDNOS_1_6_ST25_CIP1.txt created on Sep. 7, 2021, and is 2 KB in size. The sequence listing is incorporated by reference as if fully recited herein.

### BACKGROUND

Described herein are systems, devices, compositions, and methods for diagnosing and treating diseases, and more specifically to compositions and methods for diagnosing infections such as acute infectious peritonitis in patients undergoing chronic peritoneal dialysis.

Peritoneal dialysis is a well-established modality of chronic renal replacement therapy worldwide, accounting for 11% of the overall dialysis population [1]. The duration of chronic peritoneal dialysis is frequently limited by episodes of infectious peritonitis. Several risk factors leading to peritonitis have been addressed by modifying peritoneal dialysis catheter design and surgical techniques during catheter insertion [2]. Additional changes in connection techniques, catheter exit-site care, and patient training have reduced peritonitis rates over the last decade [3]-[5]. However, despite these improvements, peritonitis is a principal reason for hospitalization, conversion to hemodialysis, and death in peritoneal dialysis patients [6], [7]. Preventing peritoneal dialysis-related infections has recently been identified as the top priority for peritoneal dialysis patients and their caregivers [8].

Currently, the diagnosis of peritonitis relies on the identification of at least two of the following non-specific criteria: (i) clinical signs and symptoms such as abdominal pain, fever, and cloudy peritoneal fluid; (ii) peritoneal fluid leukocyte count of >100 cells/μ1 and 50% or greater neutrophils in the leukocyte differential after a 2-hour dwell time; and (iii) a positive peritoneal fluid culture. While basically effective, these criteria can sometimes lead to missed diagnosis of peritonitis as well as over-diagnosis of peritonitis. Accordingly, there is an ongoing need for more sensitive and specific diagnostic criteria for peritonitis in the peritoneal dialysis population.

### SUMMARY

The following provides a summary of certain example implementations of the disclosed inventive subject matter. This summary is not an extensive overview and is not intended to identify key or critical aspects or elements of the disclosed inventive subject matter or to delineate its scope. However, it is to be understood that the use of indefinite articles in the language used to describe and claim the disclosed inventive subject matter is not intended in any way to limit the described inventive subject matter. Rather the use of “a” or “an” should be interpreted to mean “at least one” or “one or more”.

One implementation provides a method of diagnosing infection in a patient undergoing a treatment of a predetermined type. This method comprises establishing a baseline level of at least one antimicrobial peptide in a specific patient or in a patient population undergoing treatment of a predetermined type, wherein the treatment involves accessing fluid from the patient's peritoneal cavity prior to the patient presenting with a suspected infection; measuring the level of the at least one antimicrobial peptide in a sample taken from the patient after the patient presents with a suspected infection; comparing the baseline level of the at least one antimicrobial peptide to the measured level of the at least one antimicrobial peptide; and diagnosing the patient with an infection if the baseline level of the at least one antimicrobial peptide exceeds the measured level of the at least one microbial peptide by a predetermined amount.

Another implementation provides a method of diagnosing and treating an infection in a patient undergoing a treatment of a predetermined type. This method comprises measuring the level of at least one antimicrobial peptide in a pre-treatment sample taken from a patient by contacting the pre-treatment sample with an anti-antimicrobial peptide antibody and detecting binding between the antimicrobial peptide and the anti-antimicrobial peptide antibody; placing the patient on a treatment of a predetermined type; measuring the level of the at least one antimicrobial peptide in a post-treatment sample taken from the patient by contacting the post-treatment sample with an anti-antimicrobial peptide antibody and detecting binding between the antimicrobial peptide and the anti-antimicrobial peptide antibody; comparing the levels of the at least one antimicrobial peptide in the pre-treatment sample and the post-treatment sample; diagnosing the patient with an infection if the amount of the at least one antimicrobial peptide in the post-treatment sample exceeds the amount of the at least one antimicrobial peptide in the pre-treatment sample by a predetermined amount; and administering an effective amount of antibiotic treatment to the patient for treating the infection. The at least one antimicrobial peptide may be selected from the ribonuclease A superfamily. The antimicrobial peptide may be RNase 3, RNase 6, RNase 7, or combinations thereof. The levels of antimicrobial peptides may be measured using quantitative antibody-based immunoassays, and the quantitative antibody-based immunoassays may include enzyme-linked immunosorbent assays and lateral flow assays. The pre-treatment sample and post-treatment sample may include peritoneal fluid, ascites fluid, or post-surgical drain fluid. The treatment may be peritoneal dialysis and the infection may be acute infectious peritonitis.

Still another implementation provides a kit for measuring ribonuclease (RNase) antimicrobial peptides in a test sample. This kit comprises reagents for extracting ribonucleic acid (RNA) from a test sample; reagents for reverse transcribing the extracted RNA into complementary deoxyribonucleic acid (cDNA); reagents for amplifying the cDNA using quantitative polymerase chain reaction (qPCR), wherein the reagents include RNase gene-specific primers; reagents for quantifying amplified RNAses, wherein the reagents include labeled antibodies that bind directly or indirectly to the amplified RNAses. The amplified RNAses may include RNase 3, RNase 6, RNase 7, or combinations thereof. The RNase gene-specific primers include the sequences of SEQ ID NO:1, SEQ ID NO:2, SEQ ID NO:3, SEQ ID NO:4, SEQ ID NO:5, and SEQ ID NO:6.

It should be appreciated that all combinations of the foregoing concepts and additional concepts discussed in greater detail below (provided such concepts are not mutually inconsistent) are contemplated as being part of the inventive subject matter disclosed herein and may be implemented to achieve the benefits as described herein. Additional features and aspects of the disclosed system, devices, and methods will become apparent to those of ordinary skill in the art upon reading and understanding the following detailed description of the example implementations. As will be appreciated by the skilled artisan, further implementations are possible without departing from the scope and spirit of what is disclosed herein. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more example implementations of the disclosed inventive subject matter and, together with the general description given above and detailed description given below, serve to explain the principles of the disclosed subject matter, and wherein:

FIGS. 1A-1D are a series of graphs depicting peritoneal fluid RNase concentrations in chronic peritoneal dialysis patients in the absence of infection and following peritonitis, wherein in FIG. 1A, in 27 uninfected, chronic peritoneal dialysis patients, RNase 7 concentrations were increased compared to RNase 3 and RNase 6 (note: the lines indicate median and interquartile range, *adjusted p<0.0001, Kruskal-Wallis test with Dunn's correction for multiple comparisons); and FIGS. 1B-D illustrate RNase 3 (FIG. 1B), RNase 6 (FIG. 1C), and RNase 7 (FIG. 1D) concentrations in 22 peritoneal dialysis patients with peritonitis, wherein each patient's RNase concentration during peritonitis is compared to that obtained in the absence of infection (“stable”;**, p<0.0001, Wilcoxon matched-pairs signed rank test);

FIGS. 2A-2E are a series of graphs illustrating increased RNase 3 expression in patients with recurrent peritonitis, wherein RNase3 levels increased with each recurrent peritonitis episode (P), compared to intervals obtained at least 3 months after peritonitis (Stable, S), and wherein FIG. 2A depicts RNase 3 (pg/ml) levels from Patient 2, FIG. 2B depicts RNase 3 (pg/ml) levels from Patient 5, FIG. 2C depicts RNase 3 (pg/ml) levels from Patient 10, FIG. 2D depicts RNase 3 (pg/ml) levels from Patient 15, and FIG. 2E depicts RNase 3 (pg/ml) levels from Patient 16;

FIGS. 3A-3C are a series of graphs that illustrate that the areas under the receiver operator characteristics (ROC) curves identify RNase 3 (FIG. 3A), RNase 6 (FIG. 3B), and RNase 7 (FIG. 3C) as potential biomarkers of peritonitis (AUC: Area under the ROC curve. * p<0.0001 and ** p=0.0012);

FIGS. 4A-4B are images illustrating that human mesothelial cells express RNASE7 mRNA and RNase 7 protein (SV40-immortalized mesothelial (Meso) cells were derived from greater omentum and extracted for RNA and protein), and wherein FIG. 4A depicts RT-PCR detection of RNASE7 mRNA in Meso cDNA (+RT) and skin, a known source of RNASE7 expression [32] (no amplification occurred in control reactions in which reverse transcriptase was omitted (−RT) or when water (W) was substituted as template); and FIG. 4B depicts detection of RNase 7 protein in mesothelial cell lysate by Western blotting (a reactive band at the expected molecular weight (15 kDa) co-migrates with recombinant human RNase 7 (rR7) and a RNase 7 reactive band from peritoneal dialysis patient 27 (Pt27));

FIGS. 5A-5F are a series of images depicting RNase expression by cells obtained from peritoneal fluid of uninfected pediatric peritoneal dialysis patients (all Figures are 40× original magnification; scale bar: 25 microns; representative images from at least 3 separate patients are shown); wherein FIG. 5A is a graph showing RNASE3, RNASE6, and RNASE7 mRNA levels (note: lines indicate median and interquartile range. # p=0.0017; Kruskal-Wallis test with Dunn's correction for multiple comparisons; FIG. 5B includes a photograph of Western blots from two patients (Pt) showing RNase 3, RNase 6, and RNase 7 protein expression in peritoneal effluent cell extracts (GAPDH is included as a loading control); and FIGS. 5C-5F are various images illustrating that RNases exhibit cell-specific distribution in peritoneal fluid (the initial drain was collected prior to starting nightly CCPD and subject to cytocentrifugation), wherein FIG. 5C is an image of Kwik-Diff staining being used to identify eosinophils (solid arrow), macrophages (dashed arrows) and mesothelial cells (arrowhead) in peritoneal fluid; FIG. 5D is an image showing granular distribution of RNase 3 in a CD66b(+) eosinophil; FIG. 5E is an image showing RNase 6 reactivity and partial co-localization with CD68, a macrophage lineage marker; and FIG. 5F is an image of cytoplasmic staining of RNase 7 in a Cytokeratin (CK)(+) cluster of mesothelial cells;

FIGS. 6A-6D are a series of images showing the localization of RNases in omentum from children with stage 5 CKD, wherein FIG. 6A is an image of Haemotoxylin and Eosin (H&E) staining illustrating normal omental morphology (arrows indicate mesothelium and abundant capillaries are evident within the mesothelium); FIG. 6B is an image of RNase 3 expressed by leukocytes (dashed arrows) in omental blood vessels; FIG. 6C is an image of RNase 6 expressed by rare interstitial cells (black arrowhead) in the submesothelial space near blood vessels (x); and FIG. 6D is an image of RNase 7 is expressed by mesothelial cells (scales bars indicate 32.5 microns (FIG. 6A) and 50 microns (FIGS. 6B-6D); original magnification 60× (FIG. 6A) and 40× (FIGS. 6B-6D); representative micrographs from 4 patients are shown);

FIGS. 7A-7C are images showing the impact of peritonitis on RNase 3 expression in pediatric omentum, wherein FIG. 7A shows sparse omental RNase 3+ cells (arrow) in peritoneal dialysis patients without a history of peritonitis; FIG. 7B shows widespread omental RNase 3+ cells following peritonitis (arrows) (both micrographs are 20× magnification; scale bars indicate 20 μm); and FIG. 7C is a graph showing quantification of RNase 3+ cell frequency in children undergoing peritoneal dialysis with a history of peritonitis, compared to indicated control populations (*, p<0.0001, Kruskal-Wallis test with Dunn's correction for multiple comparisons; 9-10 patients/group); and

FIG. 8 illustrates a network of RNases protecting the peritoneum from microbial invasion, wherein mesothelial cells constitutively secrete RNase 7 (R7), macrophages patrol the submesothelial space and synthesize RNase 6 (R6), eosinophils circulate within the peritoneal microvasculature and produce RNase 3 (R3), and both R3+ eosinophils and R6+ macrophages patrol the peritoneal cavity.

### DETAILED DESCRIPTION

Example implementations are now described with reference to the Figures. Although the following detailed description contains many specifics for purposes of illustration, a person of ordinary skill in the art will appreciate that many variations and alterations to the following details are within the scope of the disclosed implementations. Accordingly, the following implementations are set forth without any loss of generality to, and without imposing limitations upon, the claimed subject matter.

Disclosed implementations relate generally to systems, devices, compositions, and methods for diagnosing and treating diseases, particularly infectious diseases. More specifically, disclosed implementations include compositions and methods for aiding medical practitioners in early and accurate diagnosis of certain conditions such as acute peritonitis in patients undergoing peritoneal dialysis or other treatments. As described above, infectious peritonitis is a common complication in patients undergoing chronic peritoneal dialysis, limiting the duration of peritoneal dialysis as a modality for renal replacement therapy and increasing patient morbidity and mortality. In addition to the methods described previously (e.g., modifying peritoneal dialysis catheter design and surgical techniques during catheter insertion), further reductions in peritonitis rates may be achieved through strategies that enhance peritoneal innate immune defenses against invading microorganisms. Devising such strategies requires a more complete understanding of peritoneal defense mechanisms and how they are influenced by chronic kidney disease (CKD), peritoneal dialysis, and peritonitis. The peritoneum is lined by mesothelial cells that possess innate defense mechanisms including barrier function, constitutive secretion of antimicrobial mediators, and expression of pattern recognition receptors that serve as microbial sensors [9]-[11]. Mesothelial cells are assisted by patrolling leukocytes, such as eosinophils and macrophages, and together these cells form an interactive network to coordinate a rapid and efficient innate immune response [12], [13].

Innate immune cells utilize a variety of antibacterial peptides and proteins to neutralize microbial invaders, including antimicrobial peptides (AMPs) [14], [15]. AMPs may serve as novel antimicrobial agents [16]. In addition to their antimicrobial action, AMPs modulate the activity of epithelial and inflammatory cells, influencing diverse processes such as mitosis, wound healing, cytokine release, chemotaxis, protease-antiprotease balance, and redox homeostasis. Defensins, Cathelicidin (LL-37), and Neutrophil Gelatinase-Associated Lipocalin (NGAL) are examples of AMPs that are present in the peritoneal cavity. The ribonuclease (RNase) A superfamily encodes multiple cationic AMPs with potent antimicrobial activity against Gram-positive, Gram-negative, and fungal organisms [22]-[26]. These ribonucleases also exhibit broad spectrum activity against pathogens implicated in peritonitis in the peritoneal dialysis population. These antimicrobial RNases exhibit cell-specific expression patterns. Whereas RNase 3 and RNase 6 are produced by leukocytes, RNase 7 is constitutively secreted by epithelial cells of the skin, airway, and urinary tract [22], [27]. These antimicrobial RNases are believed to be present in the peritoneal cavity of patients undergoing chronic peritoneal dialysis, where they are produced by specific cell populations and exhibit unique changes in expression following peritonitis.

Disclosed implementations involve quantifying AMP levels, which increase during peritonitis, and by quantifying AMP levels, both the sensitivity and specificity of diagnosing peritonitis may be increased. The research discussed herein included measuring levels of these AMPs in peritoneal fluid from peritoneal dialysis patients in the absence of peritonitis at various times after peritonitis was diagnosed and then following completion of antibiotic treatment for peritonitis. The purpose of these studies was to establish the extent and duration of altered antimicrobial peptide levels during peritonitis for developing antimicrobial peptide threshold values for measuring specificity and sensitivity regarding the peritonitis diagnosis. This research demonstrated that patients with acute peritonitis exhibited elevated levels of antimicrobial peptides such as RNase 3 and RNase 7, compared to paired baseline samples obtained from the same patient in the absence of peritonitis. This research also indicated that recurrent peritonitis leads to increased levels of RNase 3 expression in the same patient. The disclosed method, therefore, enables the accurate diagnosis of peritonitis in the peritoneal dialysis population. A rapid, sensitive diagnosis of peritonitis facilitates appropriate antibiotic coverage and minimizes patient harm. Additionally, a specific diagnosis of peritonitis eliminates unnecessary antibiotic prescription, thereby reducing the development of antibiotic resistance.

### Results

Research described herein demonstrates that AMPS belonging to the ribonuclease (RNase) A superfamily are present in peritoneal fluid and increase during peritonitis in patients undergoing chronic peritoneal dialysis (PD). In the absence of peritonitis, RNase 3, RNase 6, and RNase 7 were detected in cell-free supernatants and viable cells obtained from peritoneal fluid of chronic PD patients. The cellular sources of these RNases were eosinophils (RNase 3), macrophages (RNase 6), and mesothelial cells (RNase 7). During peritonitis, RNase 3 increased 55-fold and RNase 7 levels increased 3-fold on average, whereas RNase 6 levels were unchanged. The areas under the receiver-operating characteristic curves for RNase 3 and RNase 7 were 0.99 (95% confidence interval (CI): 0.96-1.0) and 0.79 (95% CI: 0.64-0.93), respectively, indicating their potential as biomarkers of peritonitis. Discrete omental reservoirs of these RNases were evident in patients with end stage kidney disease prior to PD initiation, and omental RNase 3 reactive cells increased in patients undergoing PD with a history of peritonitis. The research described in detail below indicates that constitutive and inducible pools of antimicrobial RNases form a network to shield the peritoneal cavity from microbial invasion in patients undergoing chronic PD.

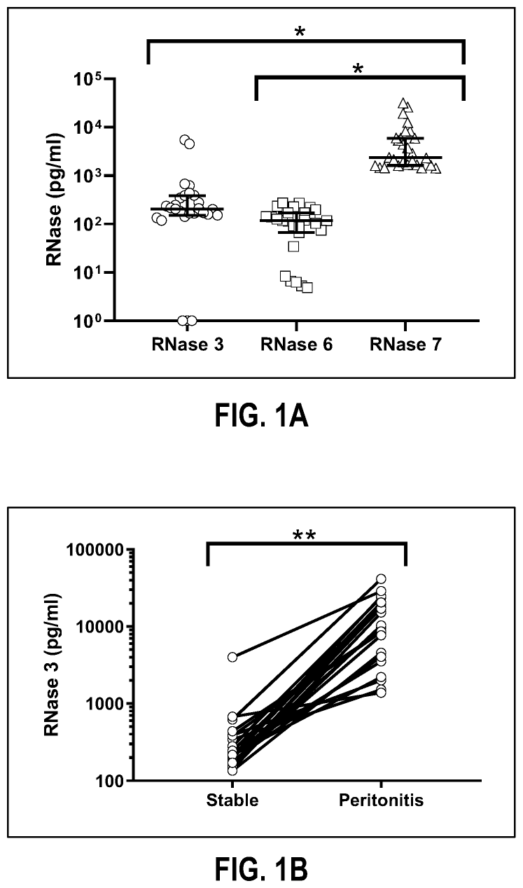

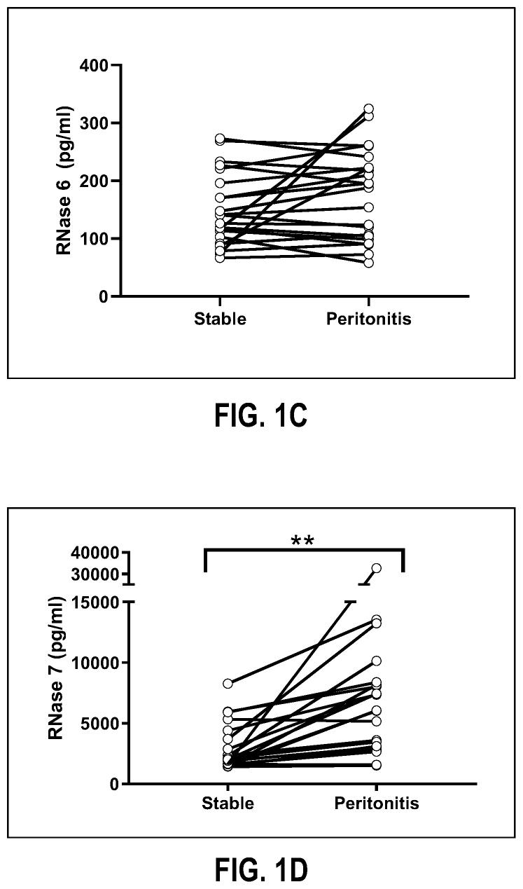

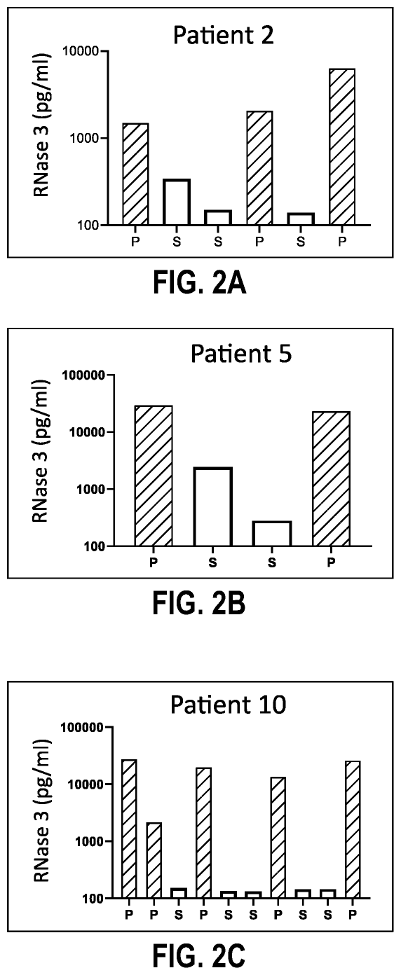

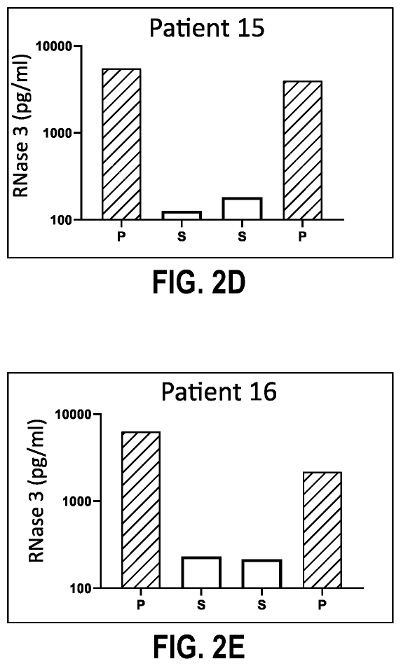

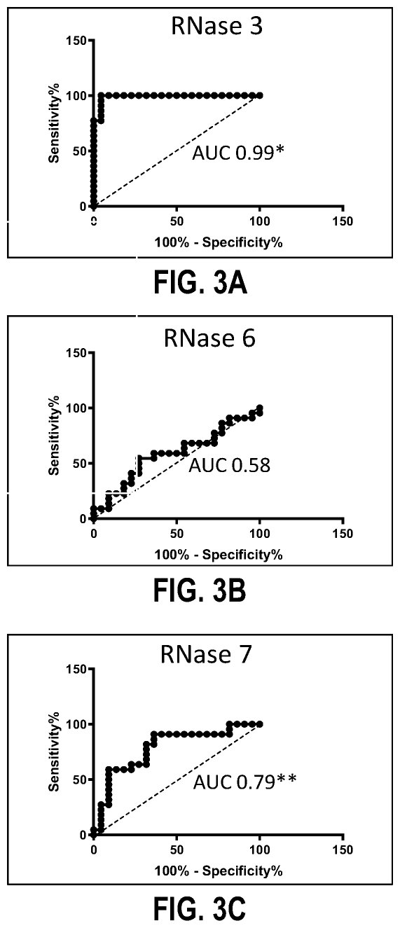

RNase 3, 6, and 7 are present in peritoneal fluid of patients on chronic PD and differentially regulated during peritonitis. Peritoneal fluid concentrations of RNase 3, 6, and 7 where investigated in a cohort of 27 patients undergoing chronic PD, comprising 21 adult and six pediatric patients (see TABLE 1, below). At the time of peritoneal fluid collection, all patients lacked signs and symptoms of peritonitis. No patient experienced a peritonitis episode within the month preceding or following peritoneal fluid collection. RNase concentrations were measured in peritoneal fluid by enzyme linked immunosorbent assay (ELISA). With reference to FIG. 1A, all three antimicrobial RNases were detectable in all patients, with the exception of three pediatric patients who lacked RNase 3 protein expression. RNase 7 levels were significantly higher than RNase 3 or RNase 6 (see FIG. 1A). RNase concentrations were then measured in peritoneal fluid samples from 21 adults and one pediatric patient with peritonitis. Concentrations were compared to uninfected samples collected from the same individuals. During peritonitis, RNase 3 levels increased 55-fold on average (range 2-134), from a median value of 211 μg/ml (IQR 167-385) in uninfected fluid to 9977 μg/ml (IQR 3906-20,332) in peritonitis see FIG. 1B). In five adult patients who experienced recurrent peritonitis, RNase 3 concentration returned to uninfected levels when measured >3 months post-infection and subsequently increased with the next peritonitis episode (see FIG. 1B). RNase 6 levels did not vary significantly between uninfected and peritonitis samples (see FIG. 1C). RNase 7 levels increased 3-fold on average (range 1-19), from a median value of 2071 μg/ml (IQR 1592-4628) in uninfected fluid to 6692 μg/ml (IQR 3080-8805) in adults with peritonitis (see FIG. 1D). The areas under the receiver operator characteristics (ROC) curves for RNase 3 and RNase 7 were 0.99 (95% CI, 0.97-1.0) and 0.79 (95% CI, 0.64-0.93), respectively (see FIG. 2). No statistically significant relationship between RNase levels and the outcome of peritoneal fluid culture (i.e., culture negative, Gram-negative, or Gram-positive bacteria) in patients with peritonitis were observed.

TABLE 1
Clinical Characteristics of Study Population.

| | | Dialysis | # Total | Episode # | | | | Age | |
|---|---|---|---|---|---|---|---|---|---|
| | Sample | vintage | peritonitis | under | | ESKD | | at PD | PD |
| Sample | type | (yr) | episodes | evaluation | Bacteriology | diagnosis | Sex | initiation | Modality |
| 1 | Peritonitis | 3.08 | 5 | 5 | Staphylococcus aureus | Focal segmental | M | 69.5 | CAPD |
| | Stable | 4.06 | | N/A | N/A | glomerulosclerosis | | | |
| 2 | Peritonitis | 2.80 | 3 | 1 | Staphylococcus aureus | Right | M | 47.2 | CAPD |
| | Stable | 3.46 | | N/A | N/A | nephrectomy | | | |
| | Stable | 3.82 | | | N/A | | | | |
| | Peritonitis | 3.89 | | 2 | Coagulase negative Staphylococcus | | | | |
| | Stable | 4.28 | | N/A | N/A | | | | |
| | Peritonitis | 4.93 | | 3 | Staphylococcus aureus | | | | |
| 3 | Peritonitis | 2.45 | 1 | 1 | Escherichia coli | Diabetic | M | 74.5 | CAPD |
| | Stable | 3.21 | | N/A | N/A | nephropathy | | | |
| 4 | Peritonitis | 1.62 | 2 | 1 | Streptococcus sanguinis | CKD, | F | 56.0 | CAPD |
| | Stable | 2.37 | | N/A | N/A | Cause not specified | | | |
| 5 | Peritonitis | 0.63 | 7 | 3 | Staphylococcus aureus | IgA nephropathy | M | 65.3 | CAPD |
| | Stable | 0.25 | | N/A | N/A | | | | |
| | Stable | 0.58 | | N/A | N/A | | | | |
| | Peritonitis | 2.25 | | 7 | Culture not sent | | | | |
| 6 | Stable | 3.51 | 2 | N/A | N/A | Diabetic nephropathy | M | 79.2 | CAPD |
| | Peritonis | 3.66 | | 1 | Escherichia coli | | | | |
| | Peritonis | 3.69 | | 2 | Culture negative | | | | |
| | Stable | 3.99 | | N/A | N/A | | | | |
| 7 | Peritonitis | 2.25 | 4 | 2 | Coagulase negative Staphylococcus | Hypersensitive | M | 75.7 | CAPD |
| | Peritonitis | 3.99 | | 3 | Coagulase negative Staphylococcus | renovascular | | | |
| | Stable | 4.31 | | N/A | N/A | disease | | | |
| | Stable | 4.38 | | N/A | N/A | | | | |
| 8 | Peritonitis | 0.94 | 2 | 1 | Enterobacter species | CKD, | M | 78.8 | CAPD |
| | Stable | 2.11 | | N/A | N/A | Cause not specified | | | |
| 9 | Peritonitis | 0.02 | 1 | 1 | Culture negative | CKD, | M | 83.9 | CAPD |
| | Stable | 1.02 | | N/A | N/A | Cause not specified | | | |
| 10 | Peritonitis | 1.07 | 5 | 1 | Coagulase negative Staphylococcus | Ischemic nephropathy | M | 74.1 | CAPD |
| | Peritonitis | 1.36 | | 2 | Culture negative | | | | |
| | Stable | 2.00 | | N/A | N/A | | | | |
| | Peritonitis | 2.23 | | 3 | Corynebacterium jeikeium | | | | |
| | Stable | 2.55 | | N/A | N/A | | | | |
| | Stable | 2.76 | | N/A | N/A | | | | |
| | Peritonitis | 2.94 | | 4 | Coagulase negative Staphylococcus | | | | |
| | Stable | 3.46 | | N/A | N/A | | | | |
| | Stable | 3.70 | | N/A | N/A | | | | |
| | Peritonitis | 2.99 | | 5 | Coagulase negative Staphylococcus | | | | |
| 11 | Peritonitis | 0.97 | 6 | 2 | Coagulase negative Staphylococcus | Traumatic or surgical | M | 72.0 | CAPD |
| | Peritonitis | 1.06 | | 3 | Coagulase negative Staphylococcus | renal loss | | | |
| | Peritonitis | 1.39 | | 4 | Staphylococcus aureus | | | | |
| | Peritonitis | 1.88 | | 5 | Coagulases negative Staphylococcus | | | | |
| | Stable | 1.97 | | N/A | N/A | | | | |
| | Stable | 2.09 | | N/A | N/A | | | | |
| | Stable | 2.40 | | N/A | N/A | | | | |
| | Stable | 3.65 | | N/A | N/A | | | | |
| 12 | Stable | 0.60 | 2 | N/A | N/A | Polycytic kidney disease | M | 82.5 | CAPD |
| | Stable | 2.38 | | N/A | N/A | (AD-unspecified) | | | |
| | Peritonitis | 3.14 | | 1 | Escherichia coli | | | | |
| | Peritonitis | 3.48 | | 2 | Escherichia coli | | | | CCPD |
| 13 | Stable | 1.29 | 1 | N/A | N/A | CKD, | F | 71.6 | CAPD |
| | Peritonitis | 1.63 | | 1 | Alpha hemolytic Streptococcus | Cause not specified | | | |
| | Stable | 2.27 | | N/A | N/A | | | | |
| 14 | Peritonitis | 0.38 | 2 | 1 | Group B Streptococcus | Idiopathic membranous | F | 58.5 | CAPD |
| | Peritonitis | 0.81 | | 2 | Neisseria species | nephropathy | | | |
| | Stable | 1.56 | | N/A | N/A | | | | |
| | Stable | 1.63 | | N/A | N/A | | | | |
| 15 | Peritonitis | 0.24 | 3 | 1 | Streptococcus sanguinis | Renal dysplasia | F | 32.8 | CAPD |
| | Stable | 0.60 | | N/A | N/A | | | | |
| | Stable | 0.75 | | N/A | N/A | | | | |
| | Peritonitis | 2.96 | | 3 | Staphylococcus aureus | | | | |
| 16 | Peritonitis | 0.80 | 2 | 1 | Escherichia coli | Polycytic kidney disease | F | 57.6 | CAPD |
| | Stable | 1.08 | | N/A | N/A | (AD-unspecified) | | | |
| | Stable | 1.50 | | N/A | N/A | | | | |
| | Peritonitis | 1.73 | | 2 | Acinetobacter ursingi | | | | |
| 17 | Peritonitis | 0.44 | 2 | 1 | Coagulase negative Staphylococcus | Ischemic nephropathy | M | 74.1 | CCPD |
| | Peritonitis | 0.52 | | 2 | Coagulase negative Staphylococcus | | | | |
| | Stable | 1.25 | | N/A | N/A | | | | |
| 18 | Peritonitis | 0.33 | 1 | 1 | Coagulase negative Staphylococcus | IgA nephropathy | M | 26.2 | CAPD |
| | Stable | 0.58 | | N/A | N/A | | | | |
| 19 | Peritonitis | 0.80 | 1 | 1 | Alpha hemolytic Streptococcus | Idiopathic membranous | F | 41.7 | CAPD |
| | Stable | 1.38 | | N/A | N/A | nephropathy | | | |
| 20 | Peritonitis | 0.07 | 2 | 1 | Alpha hemolytic Streptococcus | Diabetic nephropathy | F | 51.8 | CAPD |
| | Stable | 0.37 | | N/A | N/A | (DM2) | | | |
| 21 | Stable | 1.05 | 3 | N/A | N/A | CKD, | F | 51.5 | CAPD |
| | Peritonitis | 1.18 | | 2 | Coagulase negative Staphylococcus | Cause not specified | | | |
| 22 | Stable | 0.59 | 0 | N/A | N/A | CKD, | F | 13.8 | CCPD |
| | | | | | | Cause not specified | | | |
| 23 | Stable | 0.42 | 0 | N/A | N/A | Posterior urethral valves | M | 15.0 | CCPD |
| 24 | Stable | 0.25 | 0 | N/A | N/A | CKD, | M | 2.7 | CCPD |
| | | | | | | Cause not specified | | | |
| 25 | Stable | 5.66 | 1 | N/A | N/A | Renal cystic dysplasia | F | 4.4 | CCPD |
| 26 | Stable | 1.25 | 0 | N/A | N/A | Anti-Glomerular | M | 19.8 | CCPD |
| | | | | | | Basement Membrane | | | |
| | | | | | | Disease | | | |
| 27 | Stable | 4.08 | 3 | N/A | Never | Posterior urethral valves | M | 0.3 | CCPD |
| | Peritonitis | 5.38 | | 3 | Coagulase negative Staphylococcus | | | | |

### Cellular Sources of RNases in Peritoneal Fluid.

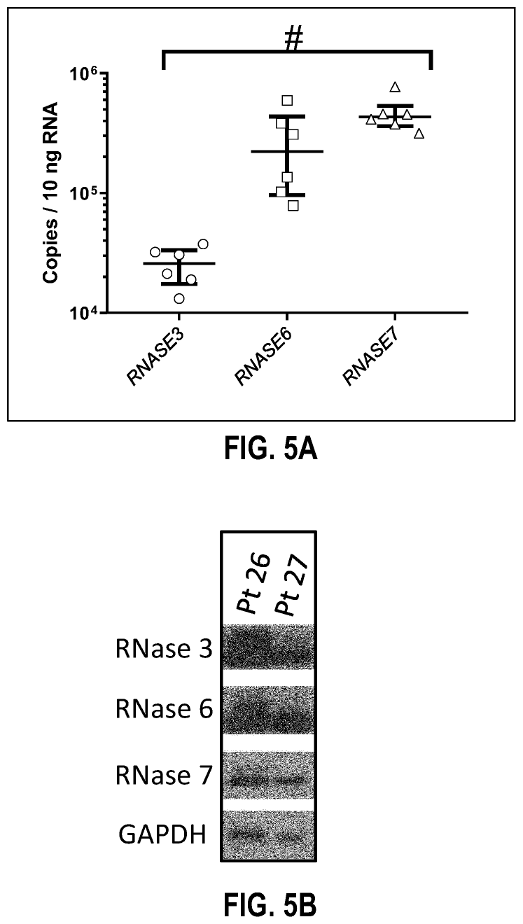

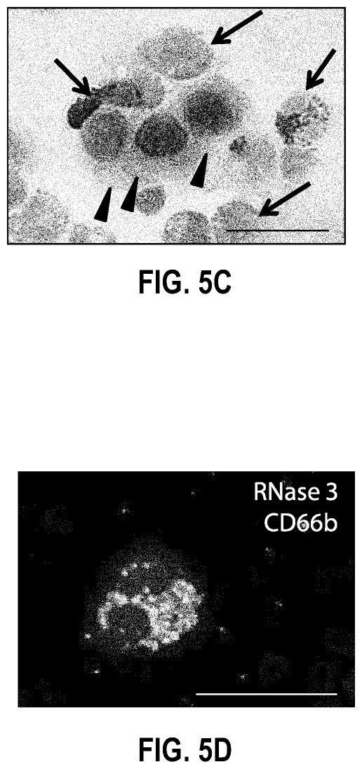

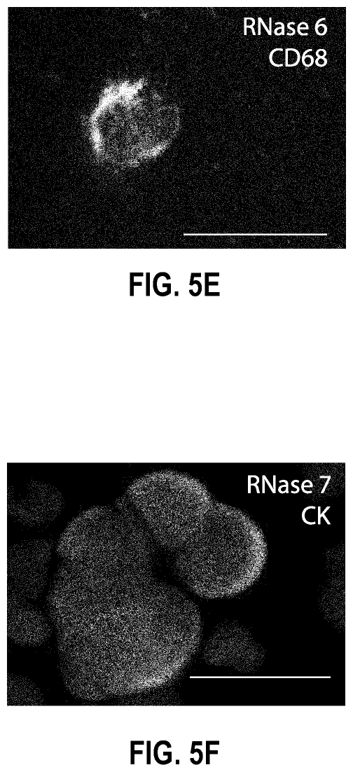

The presence of RNases in uninfected peritoneal fluid may be due to their local production by cells within the peritoneal cavity. Accordingly, viable cells were isolated from peritoneal fluid of six pediatric PD patients and RNASE3, RNASE6, and RNASE7 mRNA levels were measured by quantitative reverse transcriptase polymerase chain reaction (qRT-PCR). In all cases, RNASE mRNA expression was detectable, and RNASE7 mRNA levels were more abundant than RNASE3 (see FIG. 5A). Western blotting confirmed the presence of an intracellular pool of each RNase protein in peritoneal fluid cells (see FIG. 5B). Using a differential staining method, mesothelial cells, monocytes, and eosinophils were detected in peritoneal fluid from these patients (see FIG. 5C). RNase expression in these cells was evaluated using immunofluorescence microscopy and performed co-staining with lineage specific antibodies. RNase 3, RNase 6, and RNase 7 localized exclusively to CD66b(+) eosinophils, CD68(+) monocytes, and Cytokeratin (CK)(+) mesothelial cells, respectively (FIG. 5C).

RNases Localize to Discrete Cell Types within Human Omentum

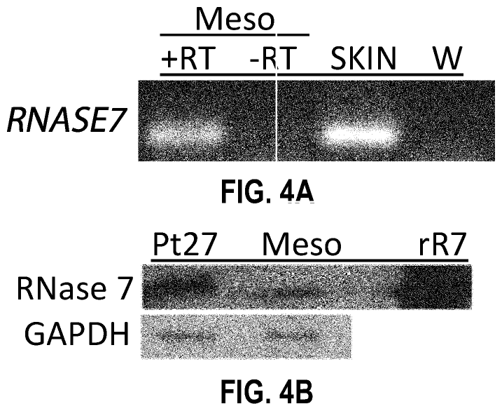

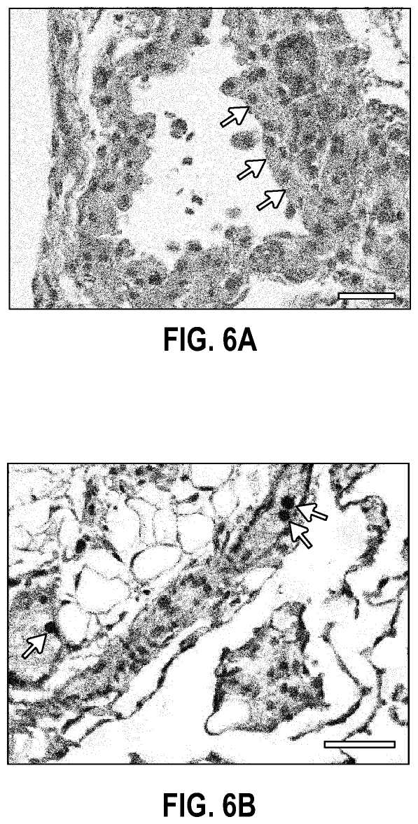

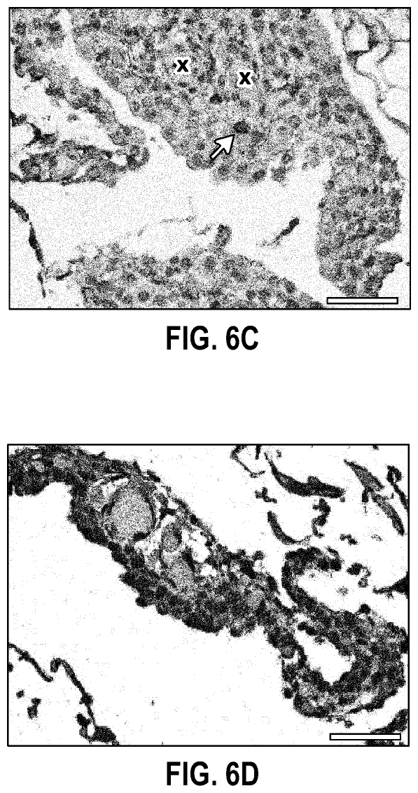

RNase distribution was investigated using immunohistochemistry in omentum from children with stage 5 CKD at the time of PD catheter insertion. The omentum is a highly vascularized structure composed of fibroadipose tissue that is lined with mesothelial cells (see FIG. 6A). RNase 3 localized to leukocytes within omental blood vessels (see FIG. 6B), while RNase 6 reactivity was restricted to occasional cells in the submesothelial space (see FIG. 6C). RNase 7 uniquely localized to the mesothelial lining (see FIG. 6D). Because RNase 7 expression has not previously been reported in mesothelial cells, RT-PCR and Western blotting was performed in extracts from SV40 immortalized mesothelial cells derived from human greater omentum and the presence of RNASE7 mRNA and RNase 7 protein, respectively, was confirmed (see FIGS. 4A-4B).

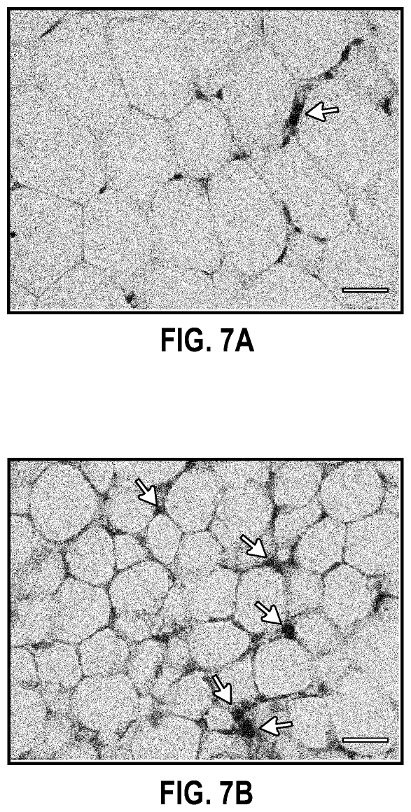

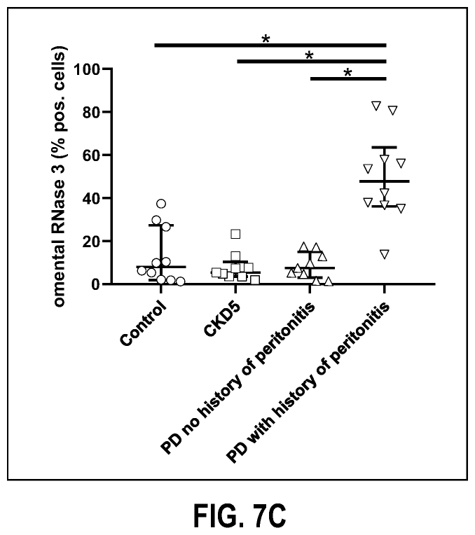

Because RNase 3 exhibited the greatest induction in peritoneal fluid during peritonitis (see FIG. 1B), the impact of peritonitis on the proportion of RNase 3 immunoreactive cells in omentum was also evaluated. For these studies, a registry of omental tissue from pediatric patients with normal kidney function, stage 5 CKD prior to dialysis initiation, and chronic PD with and without a history of peritonitis was utilized [28]. Using automated quantitative immunohistochemistry, it was determined that omentum from patients with a history of peritonitis contained a greater proportion of RNase 3+ cells, compared to the other experimental groups (see FIGS. 7A-7F).

Results indicated that AMPs in the RNase A superfamily exhibit potent, broad-spectrum antimicrobial activity toward microorganisms implicated in peritonitis in the chronic PD population [5], [24], [25], [29]. The presence of RNase 3, RNase 6, and RNase 7 in the peritoneal fluid of patients receiving chronic PD who lacked clinical signs and symptoms of peritonitis was demonstrated by this study. During peritonitis, accumulation of RNase 3 and RNase 7 in peritoneal fluid was observed. Immunolocalization studies in peritoneal fluid and omentum revealed distinct cellular reservoirs for each RNase. Collectively, these findings suggest roles for antimicrobial RNases in promoting antimicrobial immunity in patients undergoing chronic PD.

### RNase 3 Levels Increase During Peritonitis

Among the antimicrobial RNases evaluated, RNase 3 exhibited the most consistent induction during peritonitis. Increased RNase 3 levels may reflect the degranulation or recruitment of eosinophils in PD patients with peritonitis, which contain large amounts of RNase 3 in cytoplasmic granules [30]. Neutrophils represent an alternative reservoir of RNase 3, and their brisk recruitment and degranulation may account for increased RNase 3 levels in peritonitis [31]-[33]. The average concentration of RNase 3 in infected peritoneal fluid is 3-4 orders of magnitude below the minimal inhibitory concentration of recombinant RNase 3 toward Gram-positive and Gram-negative bacteria [34], [35]. It is therefore unlikely that RNase 3 exerts significant bactericidal activity in peritoneal fluid. Rather, RNase 3 may exert antimicrobial activity intracellularly, i.e., toward ingested bacteria within the eosinophil's phagosome [27].

### Mesothelial Cells are a Novel Source of RNase 7

Whereas leukocyte populations appear to account for limited RNase 3 and RNase 6 expression in peritoneal fluid, mesothelial cells serve as the sole source of RNase 7, which is the most abundant of the antimicrobial RNases evaluated in the research described herein. The production of RNase 7 by mesothelial cells is consistent with studies in the skin, airway, and urinary tract, which have demonstrated constitutive secretion of RNase7 by epithelial cells [23], [36]-[38]. These studies have led to the hypothesis that RNase 7 serves as an antimicrobial shield to prevent epithelial attachment and invasion by microorganisms [39]. Mesothelial cells express pattern recognition receptors and rapidly synthesize cytokines, chemokines, and AMPs in response to microbial exposure [40], [41]. Chronic PD leads to mesothelial cell loss, and this depletion of mesothelial cells may lead to intraperitoneal deficiency in RNase 7 and increased peritonitis susceptibility [42], [43]. Mesothelial loss as a consequence of chronic PD may account for absent induction of RNase 7 in certain patients with peritonitis. Thus, this potential link between RNase 7 levels, mesothelial content, and peritonitis susceptibility is of interest.

### Diagnostic Utility of RNase 3 and RNase 7 as Peritonitis Biomarkers

The areas under the ROC curves for RNase 7 and particularly RNase 3 predict that these ribonucleases are good to excellent candidate biomarkers of peritonitis. Peritoneal fluid cell count, differential, gram stain, and culture are the current standard evaluation tools to diagnosis peritonitis in patients receiving chronic PD. Each dialysis center is required to maintain a culture-negative peritonitis rate of <20% to ensure that the culture technique is adequate [7]; thereby highlighting the attention to detail that dialysis centers need to maintain when evaluating patients with peritonitis and the need for improved diagnostic testing. Larger scale studies would determine whether the addition of RNase 3 and RNase 7 measurement to standard evaluation methods increases the sensitivity and specificity of peritonitis diagnosis in the chronic PD patient population.

### Omentum is a Rich Source of RNase Expressing Cells

In the research described herein, omental mesothelium and leukocytes were implicated as distinct cellular sources of AMPs belonging to the RNase A superfamily. These RNases join a growing number of AMPs expressed by omentum, as omental adipocytes serve as sources of AMPs such as defensins and cathelicidin [44], [45]. When omentum was examined in patients with a history of peritonitis, a greater proportion of RNase 3+ cells—were observed even in instances where tissue was harvested 24 weeks after peritonitis. In this way, the omentum serves as a local reservoir of RNase 3+ cells that can rapidly mobilize in response to subsequent microbial exposure. The omentum is well-suited for this purpose, as omental high endothelial venules promote rapid neutrophil recruitment that protects mice with peritonitis from developing sepsis, and omental milky spots concentrate invading microorganisms where they are engulfed and killed by phagocytes [46].

The antimicrobial properties of omentum are especially intriguing in the pediatric chronic PD population, as the six pediatric patients in our study had either a partial or complete omentectomy at the time of PD catheter placement. Omentectomy is commonly performed in pediatric patients to reduce the risk of PD catheter malfunction [47]. Furthermore, pediatric PD patients represent a unique patient group to investigate the impact of PD and PD-associated peritonitis on the omentum, as the vast majority suffer from non-inflammatory diseases, mainly congenital urinary tract malformations. This lack of baseline inflammation increases the likelihood of a causal link between the observed increase in omental RNase 3+ cells and history of peritonitis in these patients.

### Methods

Adult PD Patients: This study was approved by the South East Wales Local Ethics Committee (04WSE04/27) and registered on the UK Clinical Research Network Study Portfolio under reference number #11838 “Patient immune responses to infection in PD”. All individuals provided written informed consent. 21 adults receiving continuous ambulatory PD (CAPD) or continuous cycling PD (CCPD) at the University Hospital of Wales, Cardiff, between November 2013 and March 2018 were followed for up to three years (see Samples 1-21 in Table 51, above). Cell-free peritoneal effluent samples from >8-hour dwells were collected when the individuals were stable (with no infection in the previous month), and within 24 hours of presentation with acute peritonitis, before starting antibiotic treatment. Clinical diagnosis of acute peritonitis was based on the International Society of Peritoneal Dialysis (ISPD) Consensus Guidelines [48]. Patients experienced 1-5 episodes of peritonitis during the study period. Three peritonitis episodes were defined as culture-negative (after incubation of up to 5 days), and the remaining episodes were confirmed bacterial infections. Causative microorganisms are shown in Table 51, above. Cases of fungal infection and polymicrobial or unclear culture results were excluded from the study.

Pediatric PD Patients: Following IRB approval (IRB16-00086) and provision of informed consent, we recruited six chronic PD patients at Nationwide Children's Hospital between March 2016 and September 2017 (see samples 22-27 in Table 51, above). All patients underwent CCPD using dextrose-based (high-GDP) PD solution (Dianeal, Baxter, Deerfield, Ill.). Peritoneal fluid was collected from the patients after a long (>8 hours) daytime dwell cycle prior to CCPD. All patients lacked signs and symptoms of peritonitis within the month preceding sample collection. Clinical diagnosis of acute peritonitis was based on the International Society of Peritoneal Dialysis (ISPD) Consensus Guidelines [48].

Peritoneal Fluid Processing: Following centrifugation of peritoneal fluid (500 g, 10 minutes, 4° C.), the supernatant was stored at −80° C. for ELISA. The cellular pellet was resuspended in sterile phosphate buffered saline and enumerated. Over 95% of cells recovered from PD fluid were viable based on trypan blue exclusion. The total number of live cells varied between 50,000 to 200,000. Cells were subsequently lysed in RNA or protein extraction buffer and frozen at −80° C.

Pediatric Omentum: Formalin-fixed, paraffin-embedded, deidentified human omentum from children with stage 5 CKD was provided with approval from NCH IRB in collaboration with the Department of Anatomic Pathology (Dr. Peter Baker). Alternatively, as designated in the text, omental samples from children with normal renal function (control), CKDS at the time of PD start and PD were obtained from the International Pediatric Biopsy Registry (www.clinicaltrials.gov NCT01893710). The groups were age matched. All patients received neutral pH, low GDP PD fluids. Peritonitis was caused by Staphylococcus aureus (6 cases), Enterobacter species (1 case), Corynebacterium species (1 case), and 2 cases were culture negative.

qRT-PCR: Total RNA was extracted from using the RNeasy Mini Kit (Qiagen). RNA was quantified based on absorbance at 260 nm and reverse transcribed into complementary (c)DNA with the Verso cDNA Synthesis Kit (Thermo Fisher Scientific, Waltham, Mass.) in 20 μl total volume. The cDNA was diluted with 40 μl sterile water, and 2 μl was used as template in qPCR reactions using SybrGreen detection and the following gene-specific primers: RNASE3 Forward 5′-AGA GAC TGG GAA ACA TGG-3′ (SEQ ID NO: 1); RNASE3 Reverse 5′-GAT AAT TGT TAA TTG CCC GC-3′ (SEQ ID NO:2); RNASE6 Forward 5′-AGC CCC AAC ACT GAG ACC AGA AAA-3′ (SEQ ID NO:3); RNASE6 Reverse 5′-GGT GGC AGT TGT GCC GAC GA-3′ (SEQ ID NO:4); RNASE7 Forward 5′-AAG ACC AAG CGC AAA GCG AC-3′ (SEQ ID NO:5), and RNASE7 Reverse 5′-GCA GGC TAT TTT GGG GGT CT-3′ (SEQ ID NO:6). Exclusion of reverse transcriptase during cDNA synthesis resulted in undetectable amplification, attesting to amplicon derivation from cDNA rather than contaminating genomic DNA. Positive controls consisted of bone marrow cDNA (RNASE3, RNASE6) and skin cDNA (RNASE7). Amplicons were cloned into pCR4 (Invitrogen, Carlsbad, Calif.) and bidirectionally sequenced. Standard curves for each amplicon were included with each set of reactions. Absolute transcript levels were expressed per 10 ng total input RNA.

Western blotting: Total protein was extracted, and Western blots were performed as previously described. The following rabbit polyclonal antibodies were used: RNase 3 (Abcam, Cambridge, Mass.), RNase 6 (Cloud-Clone, Katy, Tex.), and RNase 7 (Sigma-Aldrich, St. Louis, Mo.).

ELISA: Commercial ELISAs were used for detection of RNase 3 (MBL International, Woburn, Mass.), RNase 6 (Cloud-Clone), and RNase 7 (Cloud-Clone) in cell-free supernatants of peritoneal fluid. The supplied protocol was followed without modification. Samples were run in triplicate. Only values falling on the standard curve were used. The lower limit of detection was 125 μg/ml (RNase 3), 6.5 μg/ml (RNase 6), and 540 μg/ml (RNase 7). The various products and reagents described herein may be provided in one or more kits configured for commercial distribution and use.

Cytology and Immunocytochemistry: 50,000 cells were subject to cytocentrifugation. Slides were differentially stained (Kwik-Diff, ThermoFisher Scientific, Waltham, Mass.). For immunostaining, cells were permeabilized with 100% cold acetone for 2 minutes at room temperature. Slides were washed in phosphate buffered saline (PBS) with 0.05% Tween-20, treated 10 minutes with Superblock (Scytek, Logan, Utah), and incubated with primary antibodies overnight at room temperature: α-RNase3 (Abcam), α-RNase6 (Cloud-Clone), α-RNase7 (Sigma), α-CD66b (Stem Cell Technologies, Vancouver, BC), α-CD68 (Stem Cell Technologies), and α-Cytokeratin (CAM5.2, Becton Dickinson, Franklin Lakes, N.J.). The next day, slides were washed, hybridized with Alexafluor 488 and Alexafluor 595 conjugated secondary antibodies (Jackson ImmunoResearch, West Grove, Pa.) for 90 minutes at room temperature, washed again, and cover slipped in mounting medium with DAPI for nuclear visualization (Vector Labs, Burlingame, Calif.). Slides were visualized using an Olympus BX51 microscope and CX9000 camera. Controls consisted of irrelevant primary and secondary only conditions.

Immunohistochemistry: Three- or four-micron sections of human omentum were subject to immunohistochemistry using the aforementioned primary antibodies, as previously described. To quantify RNase 3 reactive cells in omentum, slides were scanned at 20× magnification (NanoZoomer, Hamamatsu, Japan) and analyzed with Aperio ImageScope software (Leica, Wetzlar, Germany). Large vessels were excluded. The Positive Pixel Count Algorithm was used to determine the fraction of RNase3+ cells among total cell nuclei, expressed as a percentage.

Statistical Analysis: Indicated statistical analyses were performed using GraphPad (La Jolla, Calif.). In all cases, p<0.05 was considered statistically significant.

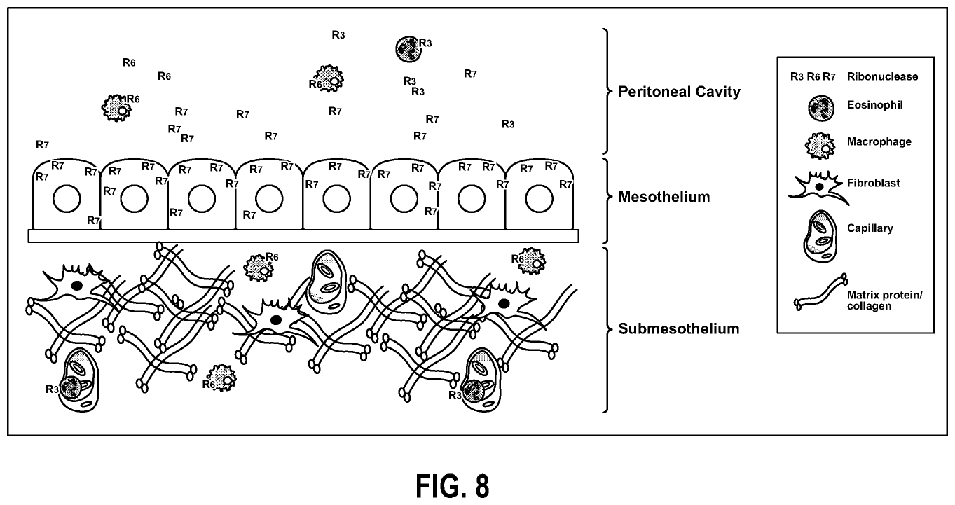

The research described herein demonstrates the presence of multiple antimicrobial RNases with distinct cellular sources in the peritoneal fluid of patients undergoing chronic PD, which display unique expression patterns following peritonitis. Peritoneal fluid concentrations of RNase 3 and RNase 7 increase during peritonitis and, therefore, these ribonucleases are effective as peritonitis biomarkers. The data support a model in which a network of RNases helps to protect the peritoneal cavity from microbial invaders (see FIG. 8). Mesothelial cells constitutively synthesize RNase 7, which acts as an antimicrobial shield at the surface of the peritoneal membrane. Sentinel leukocytes patrolling the peritoneal cavity and omentum express RNase 3 (eosinophils) and RNase 6 (monocytes/macrophages). This local antimicrobial RNase network is likely to contribute substantially to the prevention and eradication of infectious peritonitis in the PD population.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated references and similar materials differs from or contradicts this application, including but not limited to defined terms, term usage, described techniques, or the like, this application controls.

As previously stated and as used herein, the singular forms “a,” “an,” and “the,” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps. Although many methods and materials similar or equivalent to those described herein can be used, particular suitable methods and materials are described herein. Unless context indicates otherwise, the recitations of numerical ranges by endpoints include all numbers subsumed within that range. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

The terms “substantially” and “about” used throughout this specification are used to describe and account for small fluctuations, such as due to variations in processing. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%, and/or 0%.

Underlined and/or italicized headings and subheadings are used for convenience only, do not limit the disclosed subject matter, and are not referred to in connection with the interpretation of the description of the disclosed subject matter. All structural and functional equivalents to the elements of the various implementations described throughout this disclosure that are known or later come to be known to those of ordinary skill in the art are expressly incorporated herein by reference and intended to be encompassed by the disclosed subject matter. Moreover, nothing disclosed herein is intended to be dedicated to the public regardless of whether such disclosure is explicitly recited in the above description.

There may be many alternate ways to implement the disclosed inventive subject matter. Various functions and elements described herein may be partitioned differently from those shown without departing from the scope of the disclosed inventive subject matter. Generic principles defined herein may be applied to other implementations. Different numbers of a given module or unit may be employed, a different type or types of a given module or unit may be employed, a given module or unit may be added, or a given module or unit may be omitted.

It should be appreciated that all combinations of the foregoing concepts and additional concepts discussed in greater detail herein (provided such concepts are not mutually inconsistent) are contemplated as being part of the disclosed inventive subject matter. In particular, all combinations of claimed subject matter appearing at the end of this disclosure are contemplated as being part of the inventive subject matter disclosed herein. While the disclosed inventive subject matter has been illustrated by the description of example implementations, and while the example implementations have been described in certain detail, there is no intention to restrict or in any way limit the scope of the appended claims to such detail. Additional advantages and modifications will readily appear to those skilled in the art. Therefore, the disclosed inventive subject matter in its broader aspects is not limited to any of the specific details, representative devices and methods, and/or illustrative examples shown and described. Accordingly, departures may be made from such details without departing from the spirit or scope of the general inventive concept.

The following references form part of the specification of the present application and each reference is incorporated by reference herein, in its entirety, for all purposes.
1. Grassmann A, Gioberge S, Moeller S, Brown G. ESRD patients in 2004: global overview of patient numbers, treatment modalities and associated trends. Nephrol Dial Transplant. 2005; 20(12): 2587-2593.
2. Campbell D J, Johnson D W, Mudge D W, Gallagher M P, Craig J C. Prevention of peritoneal dialysis-related infections. Nephrol Dial Transplant. 2015; 30(9):1461-1472.
3. Chang J H, Oh J, Park S K, et al. Frequent patient retraining at home reduces the risks of peritoneal dialysis-related infections: A randomized study. Sci Rep. 2018; 8(1):12919.
4. Piraino B, Bernardini J, Brown E, et al. ISPD position statement on reducing the risks of peritoneal dialysis-related infections. Perit Dial Int. 2011; 31(6):614-630.
5. Sethna C B, Bryant K, Munshi R, et al. Risk Factors for and Outcomes of Catheter-Associated Peritonitis in Children: The SCOPE Collaborative. Clin J Am Soc Nephrol. 2016; 11(9): 1590-1596.
6. Mujais S. Microbiology and outcomes of peritonitis in North America. Kidney Int Suppl. 2006(103): S55-62.
7. Warady B A, Bakkaloglu S, Newland J, et al. Consensus guidelines for the prevention and treatment of catheter-related infections and peritonitis in pediatric patients receiving peritoneal dialysis: 2012 update. Perit Dial Int. 2012; 32 Suppl 2: S32-86.
8. Manera K E, Johnson D W, Craig J C, et al. Patient and Caregiver Priorities for Outcomes in Peritoneal Dialysis: Multinational Nominal Group Technique Study. Clin J Am Soc Nephrol. 2018.
9. Park J H, Kim Y G, Shaw M, et al. Nod1/RICK and TLR signaling regulate chemokine and antimicrobial innate immune responses in mesothelial cells. Journal of immunology. 2007; 179(1):514-521.
10. Yung S, Chan T M. Mesothelial cells. Peritoneal dialysis international: journal of the International Society for Peritoneal Dialysis. 2007; 27 Suppl 2: S110-115.
11. Yung S, Chan T M. Pathophysiological changes to the peritoneal membrane during PD-related peritonitis: the role of mesothelial cells. Mediators of inflammation. 2012; 2012:484167.
12. Holmes C, Lewis S. Host defense mechanisms in the peritoneal cavity of continuous ambulatory peritoneal dialysis patients. 2. Humoral defenses. Peritoneal dialysis international: journal of the International Society for Peritoneal Dialysis. 1991; 11(2):112-117.
13. Lewis S, Holmes C. Host defense mechanisms in the peritoneal cavity of continuous ambulatory peritoneal dialysis patients. 1. Peritoneal dialysis international: journal of the International Society for Peritoneal Dialysis. 1991; 11(1):14-21.
14. Ganz T. The role of antimicrobial peptides in innate immunity. Integrative and comparative biology. 2003; 43(2):300-304.
15. Wang G, Li X, Wang Z. APD2: the updated antimicrobial peptide database and its application in peptide design. Nucleic acids research. 2009; 37(Database issue): D933-937.
16. Nuti R, Goud N S, Saraswati A P, Alvala R, Alvala M. Antimicrobial Peptides: A Promising Therapeutic Strategy in Tackling Antimicrobial Resistance. Current medicinal chemistry. 2017; 24(38):4303-4314.
17. Guilhelmelli F, Vilela N, Albuquerque P, Derengowski Lda S, Silva-Pereira I, Kyaw C M. Antibiotic development challenges: the various mechanisms of action of antimicrobial peptides and of bacterial resistance. Frontiers in microbiology. 2013; 4:353.
18. Bacchetta J, Chun R F, Gales B, et al. Antibacterial responses by peritoneal macrophages are enhanced following vitamin D supplementation. PloS one. 2014; 9(12): e116530.
19. Leung J C, Lam M F, Tang S C, et al. Roles of neutrophil gelatinase-associated lipocalin in continuous ambulatory peritoneal dialysis-related peritonitis. Journal of clinical immunology. 2009; 29(3):365-378.
20. Martino F, Filippi I, Giavarina D, et al. Neutrophil gelatinase-associated lipocalin in peritoneal effluent: evaluation in peritoneal dialysis patients in basal condition. Peritoneal dialysis international: journal of the International Society for Peritoneal Dialysis. 2013; 33(4):379-381.
21. Zarrinkalam K H, Leavesley D I, Stanley J M, Atkins G J, Faull U. Expression of defensin antimicrobial peptides in the peritoneal cavity of patients on peritoneal dialysis. Peritoneal dialysis international: journal of the International Society for Peritoneal Dialysis. 2001; 21(5):501-508.
22. Becknell B, Eichler T E, Beceiro S, et al. Ribonucleases 6 and 7 have antimicrobial function in the human and murine urinary tract. Kidney international. 2015; 87(1):151-161.
23. Becknell B, Spencer J D. A Review of Ribonuclease 7's Structure, Regulation, and Contributions to Host Defense. International journal of molecular sciences. 2016; 17(3):423.
24. Rosenberg H F. RNase A ribonucleases and host defense: an evolving story. Journal of leukocyte biology. 2008; 83(5):1079-1087.
25. Boix E, Nogues M V. Mammalian antimicrobial proteins and peptides: overview on the RNase A superfamily members involved in innate host defence. Mol Biosyst. 2007; 3(5):317-335.
26. Lu L, Li J, Moussaoui M, Boix E. Immune Modulation by Human Secreted RNases at the Extracellular Space. Front Immunol. 2018; 9:1012.
27. Lehrer R I, Szklarek D, Barton A, Ganz T, Hamann K J, Gleich G J. Antibacterial properties of eosinophil major basic protein and eosinophil cationic protein. Journal of immunology. 1989; 142(12):4428-4434.
28. Schaefer B, Bartosova M, Macher-Goeppinger S, et al. Quantitative Histomorphometry of the Healthy Peritoneum. Sci Rep. 2016; 6:21344.
29. Chadha V, Schaefer F S, Warady B A. Dialysis-associated peritonitis in children. Pediatric nephrology. 2010; 25(3):425-440.
30. Olsson I, Venge P, Spitznagel J K, Lehrer R I. Arginine-rich cationic proteins of human eosinophil granules: comparison of the constituents of eosinophilic and neutrophilic leukocytes. Lab Invest. 1977; 36(5):493-500.
31. Monteseirin J, Vega A, Chacon P, et al. Neutrophils as a novel source of eosinophil cationic protein in IgE-mediated processes. J Immunol. 2007; 179(4):2634-2641.
32. Ribas D, Fernandez-Carranco M C, Hajji N, Bobadilla P, Monteseirin J. Eosinophil cationic protein and histamine production by neutrophils from patients with periodontitis. J Periodontol. 2018; 89(2):228-234.
33. Sur S, Glitz D G, Kita H, et al. Localization of eosinophil-derived neurotoxin and eosinophil cationic protein in neutrophilic leukocytes. J Leukoc Biol. 1998; 63(6):715-722.
34. Torrent M, Badia M, Moussaoui M, Sanchez D, Nogues M V, Boix E. Comparison of human RNase 3 and RNase 7 bactericidal action at the Gram-negative and Gram-positive bacterial cell wall. FEBS J. 2010; 277(7):1713-1725.
35. Torrent M, Pulido D, Valle J, Nogues M V, Andreu D, Boix E. Ribonucleases as a host-defence family: evidence of evolutionarily conserved antimicrobial activity at the N-terminus. Biochem J. 2013; 456(1):99-108.
36. Abtin A, Eckhart L, Mildner M, et al. Degradation by stratum corneum proteases prevents endogenous RNase inhibitor from blocking antimicrobial activities of RNase 5 and RNase 7. The Journal of investigative dermatology. 2009; 129(9):2193-2201.
37. Amatngalim G D, van Wijck Y, de Mooij-Eijk Y, et al. Basal cells contribute to innate immunity of the airway epithelium through production of the antimicrobial protein RNase 7. Journal of immunology. 2015; 194(7): 3340-3350.
38. Spencer J D, Schwaderer A L, Dirosario J D, et al. Ribonuclease 7 is a potent antimicrobial peptide within the human urinary tract. Kidney international. 2011; 80(2): 174-180.
39. Zasloff M. The antibacterial shield of the human urinary tract. Kidney Int. 2013; 83(4):548-550.
40. Hussain T, Nasreen N, Lai Y, Bellew B F, Antony V B, Mohammed K A. Innate immune responses in murine pleural mesothelial cells: Toll-like receptor-2 dependent induction of beta-defensin-2 by staphylococcal peptidoglycan. Am J Physiol Lung Cell Mol Physiol. 2008; 295(3): L461-470.
41. McGuire A L, Mulroney K T, Carson C F, Ram R, Morahan G, Chakera A. Analysis of early mesothelial cell responses to Staphylococcus epidermidis isolated from patients with peritoneal dialysis-associated peritonitis. PLoS One. 2017; 12(5): e0178151.
42. Schaefer B, Bartosova M, Macher-Goeppinger S, et al. Neutral pH and low-glucose degradation product dialysis fluids induce major early alterations of the peritoneal membrane in children on peritoneal dialysis. Kidney Int. 2018; 94(2):419-429.
43. Williams J D, Craig K J, Topley N, et al. Morphologic changes in the peritoneal membrane of patients with renal disease. J Am Soc Nephrol. 2002; 13(2):470-479.
44. Chandra A, Srivastava R K, Kashyap M P, Kumar R, Srivastava R N, Pant A B. The anti-inflammatory and antibacterial basis of human omental defense: selective expression of cytokines and antimicrobial peptides. PloS one. 2011; 6(5): e20446.
45. Paslakis G, Keuneke C, Groene H J, Schroppel B, Schmid H, Schloendorff D. The putative role of human peritoneal adipocytes in the fight against bacteria: synthesis of the antimicrobial active peptide DEFA1-3. Nephron Experimental nephrology. 2010; 115(4): e96-100.
46. Buscher K, Wang H, Zhang X, et al. Protection from septic peritonitis by rapid neutrophil recruitment through omental high endothelial venules. Nat Commun. 2016; 7:10828.
47. Ladd A P, Breckler F D, Novotny N M. Impact of primary omentectomy on longevity of peritoneal dialysis catheters in children. American journal of surgery. 2011; 201(3):401-404; discussion 404-405.
48. Li P K, Szeto C C, Piraino B, et al. ISPD Peritonitis Recommendations: 2016 Update on Prevention and Treatment. Pent Dial Int. 2016; 36(5):481-508.

What is claimed:

1. A method of diagnosing infection in a patient undergoing a treatment of a predetermined type, comprising:
establishing a baseline level of at least one antimicrobial peptide in a specific patient or in a patient population undergoing treatment of a predetermined type, wherein the treatment involves accessing fluid from the patient's peritoneal cavity prior to the patient presenting with a suspected infection;
measuring the level of the at least one antimicrobial peptide in a sample taken from the patient after the patient presents with a suspected infection;
comparing the baseline level of the at least one antimicrobial peptide to the measured level of the at least one antimicrobial peptide; and
diagnosing the patient with an infection if the baseline level of the at least one antimicrobial peptide exceeds the measured level of the at least one microbial peptide by a predetermined amount.

2. The method of claim 1, wherein the at least one antimicrobial peptide is selected from the ribonuclease A superfamily.

3. The method of claim 1, wherein the at least one antimicrobial peptide is RNase 3.

4. The method of claim 1, wherein the at least one antimicrobial peptide is RNase 6.

5. The method of claim 1, wherein the at least one antimicrobial peptide is RNase 7.

6. The method of claim 1, wherein the levels of antimicrobial peptides are measured using quantitative antibody-based immunoassays that include anti-antimicrobial peptide antibodies, and that detect binding between the antimicrobial peptides and the anti-antimicrobial peptide antibodies.

7. The method of claim 6, wherein the quantitative antibody-based immunoassays include enzyme-linked immunosorbent assays and lateral flow assays.

8. The method of claim 1, wherein the treatment includes peritoneal dialysis.

9. The method of claim 1, wherein the treatment includes drainage of ascites fluid.

10. The method of claim 1, wherein the treatment includes abdominal surgery.

11. The method of claim 1, wherein the infection is acute infectious peritonitis.

12. A method of diagnosing and treating an infection in a patient undergoing a treatment of a predetermined type, comprising:
measuring the level of at least one antimicrobial peptide in a pre-treatment sample taken from a patient by contacting the pre-treatment sample with an anti-antimicrobial peptide antibody and detecting binding between the antimicrobial peptide and the anti-antimicrobial peptide antibody;
placing the patient on a treatment of a predetermined type;
measuring the level of the at least one antimicrobial peptide in a post-treatment sample taken from the patient by contacting the post-treatment sample with an anti-antimicrobial peptide antibody and detecting binding between the antimicrobial peptide and the anti-antimicrobial peptide antibody;
comparing the levels of the at least one antimicrobial peptide in the pre-treatment sample and the post-treatment sample;
diagnosing the patient with an infection if the amount of the at least one antimicrobial peptide in the post-treatment sample exceeds the amount of the at least one antimicrobial peptide in the pre-treatment sample by a predetermined amount; and
administering an effective amount of antibiotic treatment to the patient for treating the infection.

13. The method of claim 12, wherein the at least one antimicrobial peptide is selected from the ribonuclease A superfamily.

14. The method of claim 12, wherein the antimicrobial peptide is RNase 3, RNase 6, RNase 7, or combinations thereof.

15. The method of claim 12, wherein the levels of antimicrobial peptides are measured using quantitative antibody-based immunoassays, and wherein the quantitative antibody-based immunoassays include enzyme-linked immunosorbent assays and lateral flow assays.

16. The method of claim 12, wherein the pre-treatment sample and post-treatment sample include peritoneal fluid, ascites fluid, or post-surgical drain fluid.

17. The method of claim 12, wherein the treatment is peritoneal dialysis and the infection is acute infectious peritonitis.

18. A kit for measuring ribonuclease (RNase) antimicrobial peptides in a test sample, comprising:
reagents for extracting ribonucleic acid (RNA) from a test sample;
reagents for reverse transcribing the extracted RNA into complementary deoxyribonucleic acid (cDNA);
reagents for amplifying the cDNA using quantitative polymerase chain reaction (qPCR), wherein the reagents include RNase gene-specific primers; and
reagents for quantifying amplified RNAses, wherein the reagents include labeled antibodies that bind directly or indirectly to the amplified RNAses.

19. The kit of claim 18, wherein the amplified RNAses includes RNase 3, RNase 6, RNase 7, or combinations thereof.

20. The kit of claim 18, wherein the RNase gene-specific primers include the sequences of SEQ ID NO:1, SEQ ID NO:2, SEQ ID NO:3, SEQ ID NO:4, SEQ ID NO:5, and SEQ ID NO:6.


SEQUENCE LISTING (6 sequences)

SEQ ID NO: 1 - DNA, 18 bp, Artificial Sequence
  RNASE3 Forward
agagactgggaaacatgg

SEQ ID NO: 2 - DNA, 20 bp, Artificial Sequence
  RNASE3 Reverse
gataattgttaattgcccgc

SEQ ID NO: 3 - DNA, 24 bp, Artificial Sequence
  RNASE6 Forward
agccccaacactgagaccagaaaa

SEQ ID NO: 4 - DNA, 20 bp, Artificial Sequence
  RNASE6 Reverse
ggtggcagttgtgccgacga

SEQ ID NO: 5 - DNA, 20 bp, Artificial Sequence
  RNASE7 Forward
aagaccaagcgcaaagcgac

SEQ ID NO: 6 - DNA, 20 bp, Artificial Sequence
  RNASE7 Reverse
gcaggctattttgggggtct